(19) United States
(12) Patent Application Publication
(10) Pub. No.: US 2008/0051679 A1
(43) Pub. Date: Feb. 28, 2008

(54) System and method for analyzing body mass

(76) Inventor: Rosemarie D. Maljanian, Farmington, CT (US)

Correspondence Address: MICHAUD-DUFFY GROUP LLP, 306 INDUSTRIAL PARK ROAD, SUITE 206, MIDDLETOWN, CT, 06457, US

(21) Appl. No.: 11/732,760

(22) Filed: Apr. 3, 2007

Related U.S. Application Data
(60) Provisional application No. 60/788,837, filed on Apr. 3, 2006.
(60) Provisional application No. 60/790,088, filed on Apr. 7, 2006.

(51) Int. Cl. A61B 5/103

(52) U.S. Cl. 600/587

(57) ABSTRACT

A body mass diagnostic system comprising input means for entry of at least one of weight data and other data and a computing device in communication with the input means. The computing device comprising a body mass index module for generating a BMI result for evaluation by a physician and a diagnostic module for generating a diagnostic result based at least on the BMI result and direction from a physician, for at least one patient to implement.

This application claims priority from provisional application Ser. No. 60/788,837, filed Apr. 3, 2006, and provisional application Ser. No. 60/790,088, filed Apr. 7, 2006, the disclosures of which are incorporated by reference herein, in their entireties.

### FIELD OF THE INVENTION

The present invention is generally directed to a body mass analysis system and is more specifically directed to a body mass analysis system that is capable of identifying actions required to control a person's weight and trending the person's body mass index (BMI).

### BACKGROUND OF THE INVENTION

A person's health can be adversely affected if that person's weight is either much less or much greater than an ideal range. For example, there appears to be a correlation between obesity and serious chronic medical conditions such as diabetes, coronary artery disease, gastrointestinal disease and many forms of cancer. Obesity is a growing public health issue in the United States, for both adults and children. Obesity can drive health care costs up and can result in lost work productivity. In addition, anorexia is another weight related disorder which is of growing concern in the United States.

Obesity has been particularly difficult to mitigate because diagnosis has been inconsistent at the point of care, leaving managed care practitioners with little information about targeted interventions and leaving benefits managers little direction regarding allocation of resources and incentives. In addition, conventional obesity metrics have been problematic because height and weight may not be accurately and consistently captured in a format that is conducive to trending. Moreover, obesity diagnosis can be lacking when physicians manage obesity with weight, height and BMI without consideration of emotional and behavioral issues, because obesity is effected more by emotional and behavioral issues than other biometrics such as blood pressure.

Body mass index (BMI) formulas, charts and calculators have been used by individuals on a casual or informative basis to determine their BMI and to help determine whether their weight may constitute an obese or anorexic condition. The United States Department of Health and Human Services, Centers for Disease Control and Prevention provides the following formulas for calculating BMI.

English Formula:
Weight in pounds÷(height in inches)²×703=BMI
Metric Formula:
Weight in kilograms÷(height in meters)=BMI

However, such self administered use of BMI data, without intervention by trained medical personal, can be misleading because factors including a person's health and physical condition may not have been taken into appropriate consideration. Some individuals ignore self administered BMI data because of weight related emotional and/or lifestyle issues. When BMI data is not acted on appropriately, serious medical conditions and/or health risks can occur. In addition, use of a BMI by itself provides a number without any medical diagnosis or course of action for the person to follow. Moreover, casual use of BMI data outside of a formal, clinical or laboratory setting is not conducive to case identification for diagnosis and treatment, trending at point of service and at the health plan level and promoting accountability amongst patient and all members of the healthcare team.

Systems for providing values indicating normal and ranges of health indicators that may be associated with abnormal health conditions, such as the Laboratory Value System have been used for certain diseases. Systems have also been used to classify diseases. For example, the International Classification of Diseases, Ninth Revision, Clinical Modification (ICD-9-CM) is based on the World Health Organization's Ninth Revision, International Classification of Diseases (ICD-9). ICD-9-CM is used for assigning codes to diagnoses and procedures associated medical conditions and diseases. The ICD-9-CM includes a list of disease code numbers.

The American Medical Association (AMA) Current Procedural Terminology (CPT®), Fourth Edition, identifies and describes codes for reporting medical services and procedures. The CPT provides a system for describing medical, surgical, and diagnostic services. The CPT is used by physicians, and health plan administrators, medical claims processors and patients.

However, the Laboratory Value System, the CPT system and the ICD-9-CM classification system have not been rigorously used together in a laboratory setting or physician's office to facilitate the documentation of the diagnosis of obesity by the physician and thus to treat obesity and other weight related disorders.

Accordingly, there is a need to provide an economical solution to the diagnosis, documenting, transmittal and monitoring of weight related disorders. Prior art methods have relied on deploying technology to the individual patient's home and provide an uncontrolled system for transmittal of data to a physician's office. The prior art home based systems are too expensive and not cost effective except for very sick patients with severe heart failure.

There is also a need to provide a method and/or system for diagnosing body mass that is accurate, supported by medical studies, facilitates consistent diagnoses of obesity as a medical condition, specifies a course of action, is administered in a controlled manner and provides adequate trending capability both at the point of service and in large claims databases such as those used by health plan administrators. Prior art methods and systems for addressing these needs were either too informal or ineffective or a combination of both of these perceived deficiencies. Prior art methods do not report BMI in the context of a laboratory value but rather as either a stand alone value for diagnosing obesity or as a risk factor that may be a causal factor attributable to worsening the patient's diabetes or heart disease. Based on the foregoing, it is the general object of the present invention to improve upon conventional methods and systems and to overcome the problems and drawbacks of the prior art.

### SUMMARY OF THE INVENTION

One aspect of the present invention provides for a body mass diagnostic system including input means for entry of at least one of a person's weight data and other data and a computing device in communication with the input means. The computing device includes a body mass index module for calculating a body mass index (BMI) result for the person and BMI normal ranges and outside of normal ranges. The body mass diagnosis system further includes a diagnostic module for determining a diagnostic result, including at least one of a recommended medical condition of the person and a course of action for the person to implement. In one embodiment the diagnostic result includes at least one ICD code.

The body mass diagnostic system includes a trending module including a data storage device for storing successive inputs of at least one of the BMI result and the diagnostic result taken over successive periods of time. The trending module transmits the successive inputs of at least one of the BMI result and the diagnostic result to at least one of the physician and the health plan administrator and the trending module compares a first of the successive inputs to a second of the successive inputs. The trending module updates the diagnostic result, stores the updated diagnostic result in the storage device, and provides said BMI result and the updated diagnostic result to the at least one patient.

The computing device includes a certification module including operator certification data and security data. The certification module compares the certification data to the security data; and if the certification data matches at least a portion of the security data, the certification module enables an operator to continue use of the computing device.

The present invention includes a method for diagnosing body mass including the step of providing a body mass diagnostic system; input means; and a computing device in communication with the input means. The computing device includes a body mass index module and a diagnostic module. The method also includes the steps obtaining weight data from a person and transmitting the weight data to the computing device; entering other data via the input means and transmitting the other data to the computing device; calculating, by mass index module, a BMI result; and transmitting the BMI result to the diagnostic module and to a physician; and determining a diagnostic result, based at least on the BMI result and input from a physician.

In one aspect of the invention the step of entering the other data includes entering by the physician at least one CPT code. In another aspect of the present invention, the step of determining the diagnostic result includes specifying, by the physician, at least one ICD code.

In one aspect of the present invention, the method includes the steps of providing a trending module within the computing device and the trending module being in communication at least one of a physicians office and a health plan administrator, the trending module including a data storage device; and storing successive inputs of at least one of the BMI result and the diagnostic result in the data storage device; and transmitting successive inputs of at least one of the BMI result and the diagnostic result in the data storage device to at least one of a physician and a health plan administrator. The method also includes the steps of comparing the successive inputs of at least one of the BMI result and the diagnostic result in the trending module; and updating at least one of the medical condition and the course of action.

Another aspect of the present invention includes the steps of providing a certification module in communication with the input means and the computing device; uploading security data into the certification module; entering certification data of an operator into the certification module via the input means; comparing the certification data to the security data; and enabling continued operation of the computing device wherein when the certification data matches at least a portion of the security data. In one embodiment the method includes the step of installing the body mass diagnostic system in at least one of a laboratory and a physician's office.

### DESCRIPTION OF THE DRAWINGS

FIG. 1 is a schematic view of the body mass diagnostic system in accordance with the teachings of the present invention.

FIG. 2 is a flow diagram of the diagnostic module.

FIG. 3 is a schematic view of the body mass diagnostic system including reporting means.

FIG. 4 is a schematic view of the body mass diagnostic system including a trending module.

FIG. 5 is a flow diagram of the trending module.

FIG. 6 is a schematic view of the body mass diagnostic system including a certification module, an interface link and a length measurement means.

### DETAILED DESCRIPTION OF THE PREFERRED EMBODIMENT

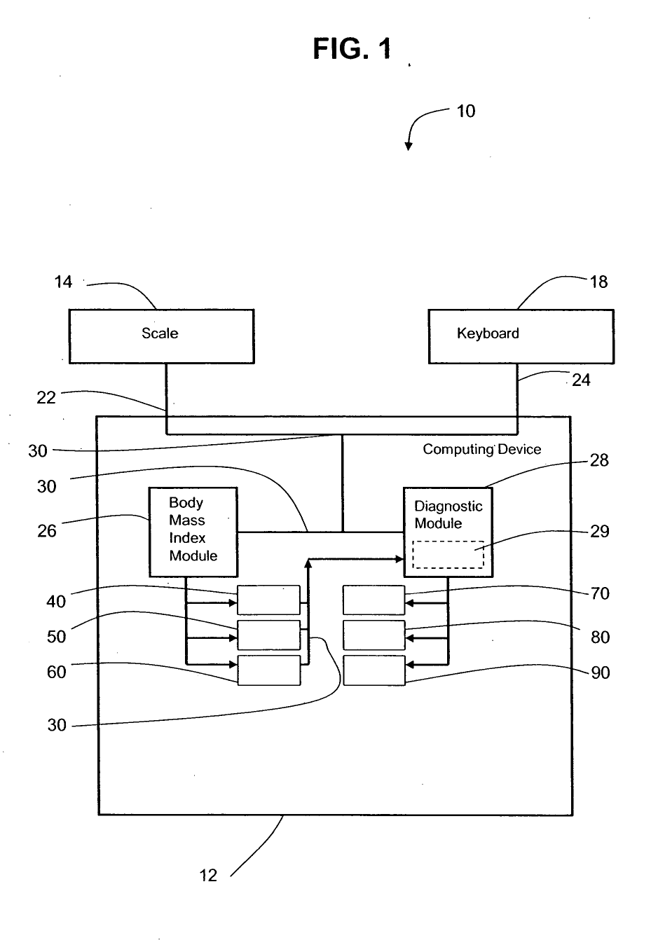

FIG. 1 illustrates a body mass diagnostic system 10 in accordance with one embodiment of the present invention. The body mass diagnostic system 10 includes a computing device 12, for example, a personal computer, workstation, laptop, tablet computer, personal digital assistant or similar portable computing device, operating within, for example, a laboratory or professional environment. The body mass diagnostic system 10 is a guide for assisting a physician to specify medical profiles and diagnose medical conditions, as described in detail below. The body mass diagnostic system 10 also includes an electronic scale 14 for obtaining weight data of a person, and an input device such as, for example, a keyboard 18 for entry of at least one of the weight data and other data. The scale 14 communicates with the computing device 12 through a first data link 22, for example, a Universal Serial Bus (USB) which transmits the weight data to the computing device. The keyboard 18 communicates with the laboratory computing device 12 through a second data link 24 for example, a USB which transmits the weight data and other data to the computing device. In one embodiment the other data comprises, for example, at least one of a height measurement of the person, a waist measurement of the person, an age of the person, gender of the person, an exercise program of the person, an athletic build of the person, body frame, a physician's orders, blood pressure, any health conditions of the person, and a date and a time corresponding to when the measurements were taken. In one embodiment, the scale is not coupled to the computing device and the weight data for the person is therefore entered with the input device, for example, the keyboard 18.

While the body mass diagnostic system has been described as including a computing device 12, the present invention is not limited in this regard as other devices can be used including but not limited to circuit boards, software, chip cards, and microprocessors which may be coupled to or installed on another device to provide the body mass diagnostic system and methods as described herein. While the keyboard 18 has been described for input of other data, the present invention is not limited in this regard as other input means can be used including but not limited to displays, touch-pad displays, images scanners, bar code readers, USB ports, voice sensing devices and the like. Although an electronic scale 14 has been illustrated for obtaining the weight data of the person, the present invention is not limited in this regard as other types of weighing devices can be used including but not limited to mechanical scales, balances, and load cells. Although the first and second data links 22, 24 are described as USB devices, the present invention is not limited in this regard as other data links may be used including but not limited to wireless communication links.

Still referring to FIG. 1, the computing device 12 includes a body mass index module 26 and a diagnostic module 28. The body mass index module 26 calculates a body mass index (BMI) of the person, reviews clinically accepted normal operating BMI ranges of below average (<18.5), average (18.5 to 24) and above average (>24), and determines whether the BMI is within or outside of the normal operating BMI ranges. The body mass index module 26 calculates the BMI of the person based on the weight data and at least a portion of the other data input via the keyboard 18. In one embodiment, the BMI is calculated in accordance with United States Department of Agriculture (USDA), Centers for Disease Control (CDC) and National Institutes of Health (NIH) guidelines or endorsed methodologies. The body mass index module 26 outputs a BMI result 40, 50, and 60 including at least one of the BMI, the normal operating BMI range, whether the BMI is within or outside of the normal operating BMI ranges, the weight data, health condition and at least a portion of the other data for a first, a second, and a third patient, respectively. Although, BMI results 40, 50, 60 are shown for three patients, the present invention is not limited in this regard as the present invention provides any number of BMI results for any number of patients. The body mass index module 26 and the diagnostic module 28 communicate with each other though a computing device link 30 of the computing device 12. The BMI module 26 transmits the BMI results to the diagnostic module 28 via the computing device link 30 for analysis and review by a physician.

Still referring to FIG. 1, the physician's orders include at least one health profile which identifies symptoms, risk factors, treatments and weight control considerations. Such health profiles include but are not limited to an obesity profile, an anorexia profile, a thyroid profile, a cardiac profile, a blood chemistry profile, a cancer profile, a diabetes profile and a genetics profile. The diagnostic module 28 includes a listing of health profiles 29 in terms of Current Procedural Terminology (CPT) Codes for selection by the physician.

The health profiles 29 include, for example, a weight management profile in which another disease is not suspected and may include but are not limited to BMI (height, weight), waist circumference, age, gender, build and body frame size. In accordance with one aspect of the present invention, if the body mass index module 26 indicates that a person is obese, a physician may order a Thyroid panel (CPT code 7020X) to rule out an underlying medical condition and a Chemistry Profile (CPT code 98142) so that if blood glucose is higher than normal and the person is obese, in addition to recommending diet, exercise and follow-up, laboratory tests such as an HbA1c test are recommended. The HbA1c test (hemoglobin A1c test, glycosylated hemoglobin A1c test, glycohemoglobin A1c test, or A1c test) is a lab test which measures average blood glucose over a period of two to three months. The HbA1c test measures the number of glucose molecules attached to hemoglobin in red blood cells over a period of time when the red blood cells, which exist for about four months, are replaced. Thus a several month history of average blood glucose levels is determined.

In the case of a Cardiac Profile 1 (CPT code 12601), which includes but is not limited to lipid studies, the effect of a weight loss regime on cholesterol levels are monitored over time and the appropriate type of medication and the dosage and timing for administration of medication is adjusted based on the impact of weight management on the cholesterol levels. Weight management profiles are assigned unique CPT codes and profiles which include BMI incorporated therein.

The diagnostic module 28 outputs a diagnostic result 70, 80, and 90 for the first, second, and third patient, respectively, based at least on the BMI result and on direction from a physician. The diagnostic result 70, 80, and 90 includes at least one of a recommended diagnosis code, at least a portion of the other data, recommended medical condition, for example a diagnosis of a medical condition, and a course of action for the first, second, and third patients to implement in conjunction with a physician. The recommended diagnosis code, the recommended medical condition and the course of action are based on evidence based medical practice guidelines. The diagnostic results 70, 80, and 90 include an International Classification of Diseases (ICD) code specified by a physician. For example one embodiment uses ICD codes from the International Classification of Diseases, Ninth Revision, Clinical Modification (ICD-9-CM) based on the World Health Organization's Ninth Revision. While the ninth revision of the ICD-9-CM is described, the present invention is not limited in this regard as other revisions and related systems can be used including earlier revisions, later revisions, ICD-9 and ICD-10.

The diagnostic result 70, 80, and 90 is specified in terms of an ICD code, associated with a weight condition. For example, the diagnostic categories include but are not limited to 1) Obesity—BMI>30, ICD code 278.00; 2) Morbid Obesity—BMI→40, ICD code 278.01; 3) Overweight BMI>25-29; 4) Pediatric Obesity, BMI>95^{th} percentile for age, ICD code V85.54 and 5) Obesity complicating pregnancy ICD code 649.00. The BMI values, CPT codes and the ICD codes are incorporated into a laboratory system reporting mechanism to facilitate diagnosis, treatment, action, and trending over time. The BMI values and the ICD codes are incorporated into infrastructure existing in healthcare systems which are used in physician offices and laboratory clinics and therefore the BMI result and the diagnostic result are provided in a format familiar to physicians and health plan administrators. In addition, the BMI result and the diagnostic result satisfy quality standards of the infrastructure existing in healthcare systems, such that physicians and health plan administrators have confidence in the use of the BMI result and the diagnostic result provided therefrom.

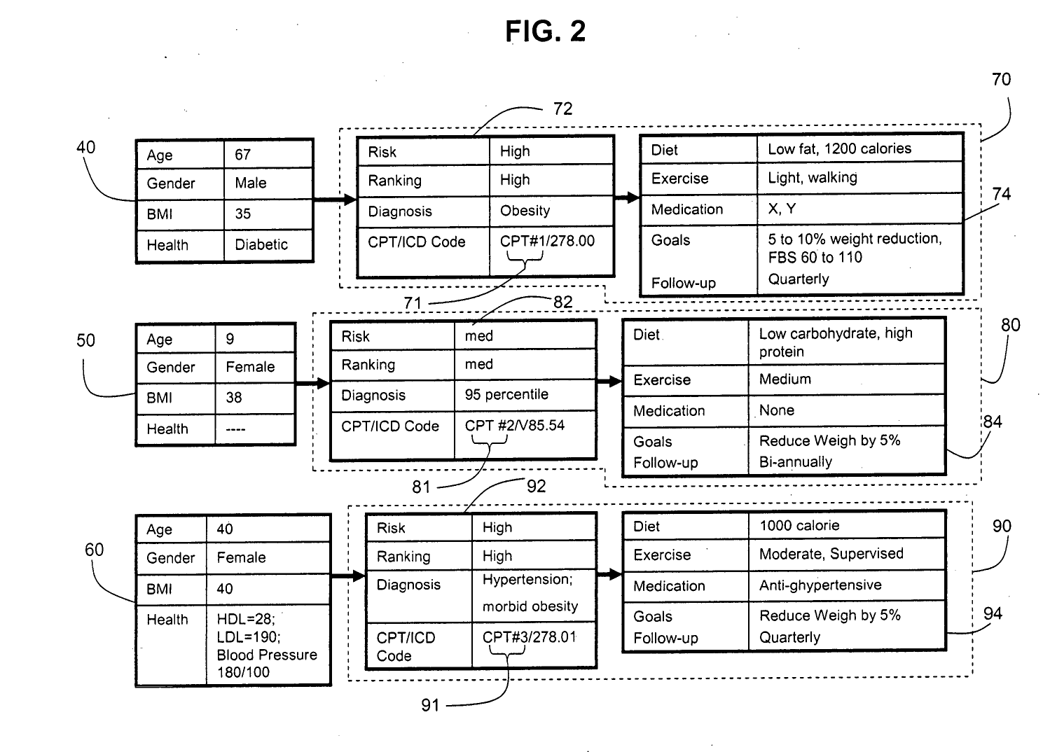

Referring now to FIG. 2, the diagnostic result 70, 80, and 90 is shown graphically for the first, second and third patients using different profiles, namely an adult chemistry profile (CPT Code #1) 71, a pediatric weight management profile (CPT Code #2) 81 and an adult cardiac profile (CPT Code #3) 91, respectively. For each of the first, second and third patients, the diagnostic module 28 outputs the BMI result 40, 50, 60 and in conjunction with input from the physician, the diagnostic module provides the diagnostic result 70, 80, and 90 including a health risk, health ranking, diagnosis, the recommended medical condition and specifies the ICD codes 72, 82, and 92 accordingly. In addition, the diagnostic result 70, 80, and 90 includes a course of action including diet, exercise, medication, goals and follow-up requirements 74, 84, 94.

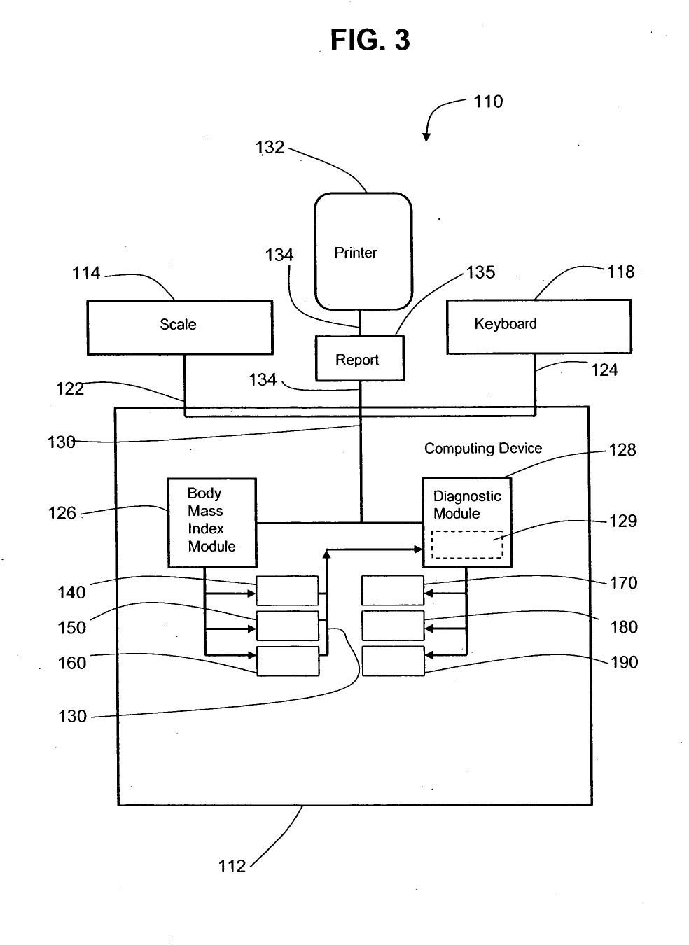

The embodiment shown in FIG. 3 is similar to that of FIG. 1, therefore like elements will be given like numbers preceded by the numeral 1. Referring to FIG. 3, in one embodiment, a body mass diagnostic system 110 includes a printer 132 in communication with computing device 112 through a third data link 134. The computing device 112 generates a report 135 including the BMI result 140, 150 and 160 of the body mass index module 126 and the diagnostic result 170, 180 and 190 of the diagnostic module 128, and transmits the report to the printer 132 for printing on paper at point of service or electronically to a patient's secure email or physician's office or health plan administrator's data file feed. The report 135 includes at least one of the weight data, at least a portion of the other data, the BMI, the medical condition and the course of action. Although the printer 132 has been described for printing the report 135, the present invention is not limited in this regard, as the report other embodiments include local and/or remote showing on a display, electronic transmission of the report, for example via e-mail, phone lines and the internet, saving report data to an electronic storage device such as a floppy or compact disc, flash memory, flash cards, memory sticks, broadcast on an audio device or a combination thereof and the printer. The computing device generates the report 135 and transmits the report to the printer for printing on paper for a patient to receive at the point of service as well as electronically via secure e-mail. Furthermore, the report 135 is transmitted to the physician based on standard laboratory reporting procedures that results in data in the report being virtually 100% reliable and therefore more likely to be reviewed by physicians compared to quality improvement reports coming from health plan administrators or other quality entities that may not be accurate and therefore can be disregarded or given low priority by physicians due to concerns of data credibility or importance.

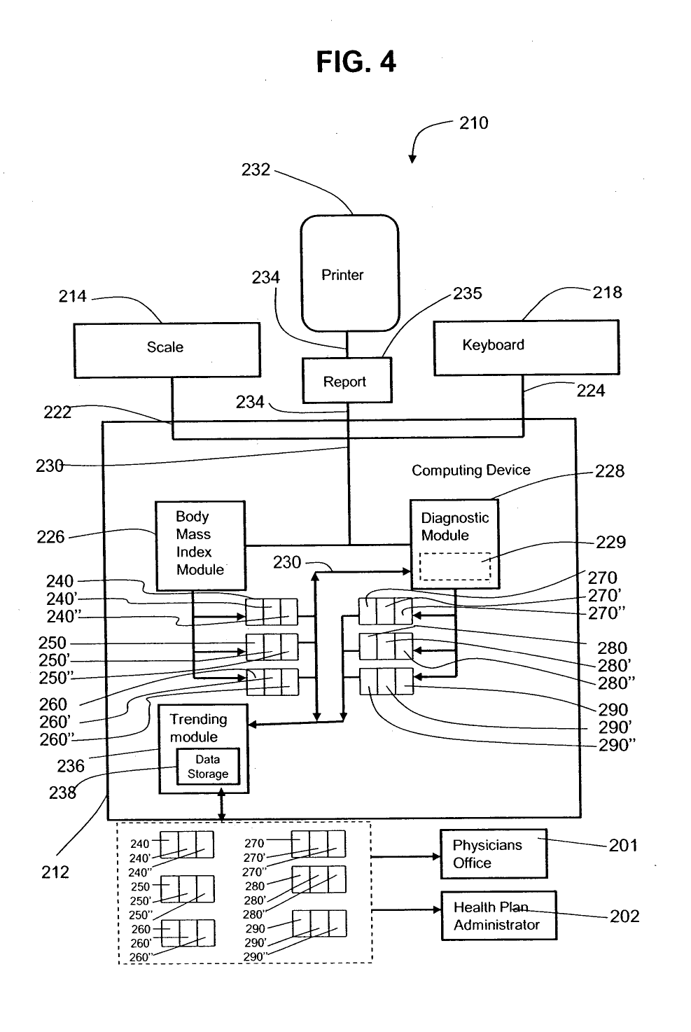

The embodiment shown in FIG. 4 is similar to that of FIG. 1, therefore like elements will be given like numbers preceded by the numeral 2. Referring to FIG. 4, in one embodiment, a body mass diagnostic system 210 comprises a computing device 212 including a trending module 236 comprising a data storage device 238 for storing successive inputs of at least one of the weight data, at least a portion of the other data, the BMI, the BMI result 240, 250, 260, the diagnostic result 270, 280, and 290 the medical condition and the course of action. It should be appreciated that successive inputs include a first set of inputs 240, 250, 260, 270, 280 and 290 at a first period of time; a second set of inputs 240′, 250′, 260′, 270′, 280′ and 290′ at a second period of time; and at least a third set of inputs 240″, 250″, 260″, 270″, 280″ and 290″ at a third period of time. Although the first, second and third sets of successive inputs are described, the present invention is not limited in this regard as any number of additional successive inputs are stored by the storage device 238. The successive inputs 240, 250, 260, 270, 280 and 290; 240′, 250′, 260′, 270′, 280′ and 290′; and 240″, 250″, 260″, 270″, 280″ and 290″ represent various intervals (dates and times) upon which patient data is collected. The trending module transmits said successive inputs of at least one of the BMI result and the diagnostic result to at least one of the physician and the health plan administrator. The trending module 236 compares the successive inputs 240, 250, 260, 270, 280 and 290; 240′, 250′, 260′, 270′, 280′ and 290′; and 240″, 250″, 260″, 270″, 280″ and 290″, accumulated over a period of time, for example monthly or quarterly, of at least one of the weight data, at least a portion of the other data, the BMI, the medical condition and the course of action. The trending module compares a first of the successive inputs to a second of said successive inputs. The trending module 236 updates at least a portion of the diagnostic result 270, 280, and 290 based on an analysis of the successive inputs 240, 250, 260, 270, 280 and 290; 240′, 250′, 260′, 270′, 280′ and 290′; and 240″, 250″, 260″, 270″, 280″ and 290″ and input from the physician and the health plan administrator and stores the updated diagnostic result in the storage device. The updated diagnostic result is provided to at least one patient. For example, if a trend is detected where the first patient's BMI is decreases but their physician has noted that their abdominal strength has not improved, the diagnostic result 270 is updated to recommend additional abdominal exercise, for example sit-ups.

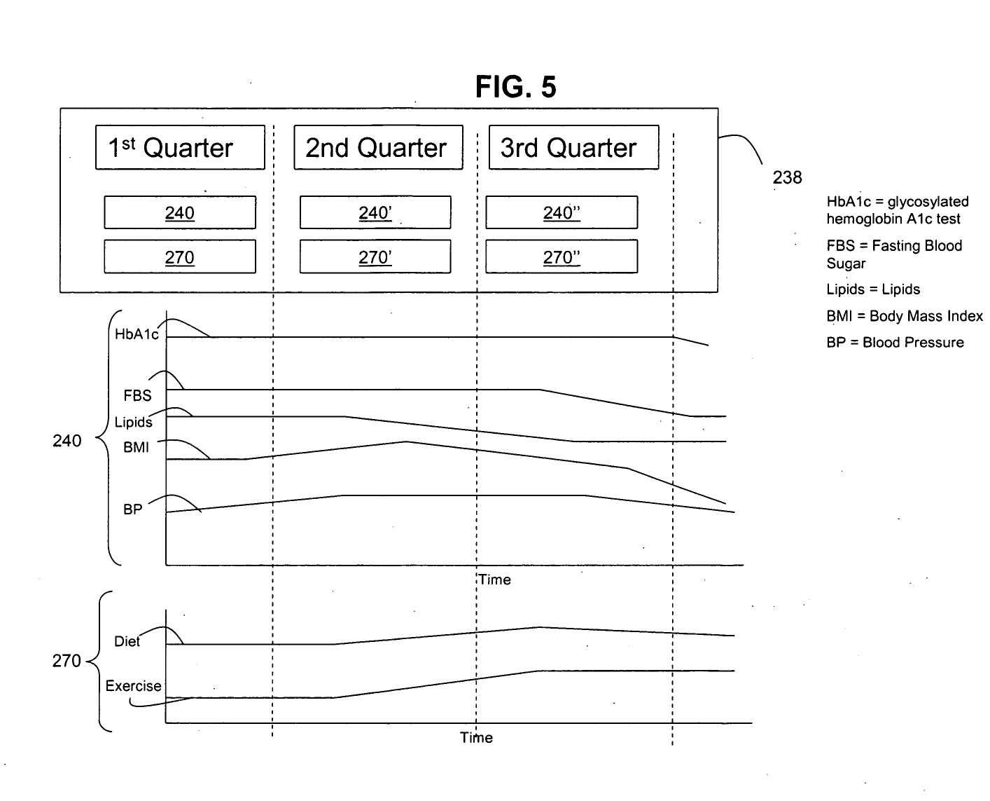

Referring to FIG. 5, in one embodiment the data storage 238 stores the successive inputs of the BMI result 240, 240′ and 240″ and of the diagnostic result 270, 270′ and 270″, for the first patient. For example, HbA1c, Fasting Blood Sugar (FBS), Lipids, BMI and Blood Pressure data is stored in the data storage 238 on a quarterly basis. Referring back to FIG. 4, the successive inputs 240, 250, 260, 270, 280 and 290; 240′, 250′, 260′, 270′, 280′ and 290′; and 240″, 250″, 260″, 270″, 280″ and 290″ are transmitted by the body mass diagnostic system 210 to at least one of a physician 201 and a health plan administrator 202. The physician 201 and/or the health plan administrator 202 provide an evaluation of the successive inputs 240, 250, 260, 270, 280 and 290; 240′, 250′, 260′, 270′, 280′ and 290′; and 240″, 250″, 260″, 270″, 280″ and 290″, and revise the diagnostic result 270, 280 and 290; 270′, 280′ and 290′; and 270″, 280″ and 290″ for the first, second and third patients respectively, for the first, second and/or third time periods. The trending module 236 adjusts the course of action, for example exercise, diet, medication and diagnosis based on evaluation of the HbA1c, Fasting Blood Sugar (FBS), Lipids, BMI and Blood Pressure data based on direction from the physician and/or health plan administrator. While the trending module is described as storing and trending HbA1c, Fasting Blood Sugar (FBS), Lipids, BMI, successive inputs of the BMI result and the diagnostic result and Blood Pressure data the present invention is not limited in this regard, as other data can be trended including but not limited to medical data and health plan administrator's data.

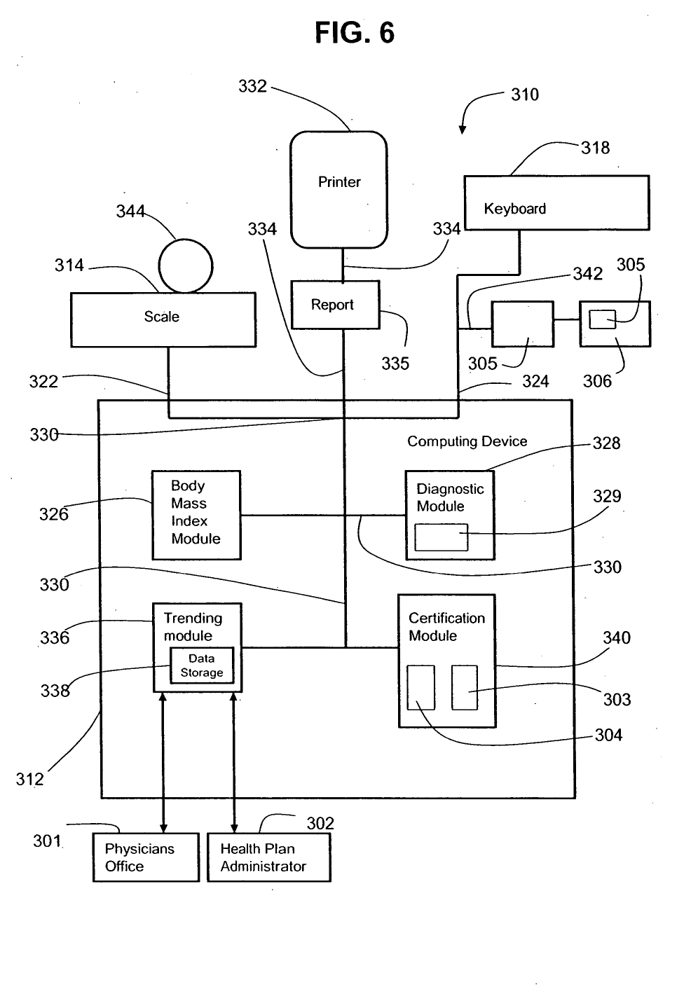

The embodiment shown in FIG. 6 is similar to that of FIG. 1, therefore like elements will be given like numbers preceded by the numeral 3. However, for clarity, the successive inputs 240, 250, 260, 270, 280 and 290; 240′, 250′, 260′, 270′, 280′ and 290′; and 240″, 250″, 260″, 270″, 280″ and 290″ are not shown. Referring to FIG. 6, in one embodiment, a body mass diagnostic system 310 includes a computing device 312 comprising a certification module 340 further comprising operator certification data 303 and security data 304. In one embodiment the operator is a person having medical or technical certification. In one embodiment, the operator certification data 303 includes a logon identification code, a password, or any data that identifies the operator as being certified or otherwise authorized to operate a body mass diagnostic system 310. The operator enters the operator certification data via the keyboard 318. The certification module 340 includes security data for validation of the certification or authority of the operator to use the body mass diagnostic system 310. The security data 304 is uploaded into the computing device 312 upon initial start-up thereof and updated as necessary thereafter. The certification module 340 compares the certification data 303 to the security data 304 and if the certification data 303 matches at least a portion of the security data 304, the certification module enables continued operation of the computing device 312. An additional level of security is also provided to ensure only certified or otherwise authorized operators can generate a report 335. Therefore a report 335 is generated if the certification data 303 matches at least an additional portion of the security data 304.

Still referring to FIG. 6, in one embodiment, the computing device 312 includes an interface link 342 between the computing device and at least one external data source 306. The at least one external data source 306 includes supplementary information 305 including at least one of medical history, laboratory reports, health plan administrator's data, self insured employer groups, third party administrators, health insurance policies and incentive plans and provides the supplementary information to the computing device 312 via the interface link 342 and the second data link 324. Referring to FIGS. 4 and 6, the diagnostic module 328 analyzes the supplementary information and modifies at least one of the successive inputs 240, 250, 260, 270, 280 and 290; 240′, 250′, 260′, 270′, 280′ and 290′; and 240″, 250″, 260″, 270″, 280″ and 290″, the medical condition, the course of action and the report 335 to include the supplemental information or consideration thereof.

Still referring to FIG. 6, in one embodiment, the scale 314 includes a tape measure 344 secured thereto for measuring the height and waist circumference of the first, second or third patient. The height and waist circumference is provided to the body mass index module 326 and the diagnostic module 328, for evaluation by the physician. The physician revises the diagnostic result 270, 280 and 290; 270′, 280′ and 290′; and 270″, 280″ and 290″ for the first, second and third patients respectively, for the first, second and/or third time periods, for example if a change in athletic build renders the BMI an inaccurate measurement of obesity for the first, second or third patient.

The present invention also includes a method for diagnosing body mass. The inventive method comprises the step of providing a body mass diagnostic system 10 comprising a scale 14; a keyboard 18; and a computing device 12 in communication with the scale and the keyboard. The computing device 12 comprises a body mass index module 26 and a diagnostic module 28. The method also includes the steps of weighing a person on the scale 14 and obtaining weight data therefrom and transmitting the weight data to the computing device 12; entering other data via the keyboard 18 and transmitting the other data to the computing device 12; the body mass index module 26 calculates a BMI result; and transmits the BMI result, weight data, the other data and the BMI to the diagnostic module 28 wherein a diagnostic result including a recommended medical condition of the person and a course of action for the person is generated based at least on the BMI result and direction from the physician for at least one patient to implement. In one embodiment, the BMI result 40, 50, and 60 includes at least one of the BMI, the normal operating BMI range, whether the BMI is within or outside of the normal operating BMI ranges, the weight data, health condition and at least a portion of the other data. This allows for physicians to consistently address the issue of obesity and other weight related conditions with their patients. When this data is viewed by a physician as part of the reports they receive from the laboratory often times along with other lab values in a diabetes or cardiac profile they are better equipped to have a discussion with the patient on diagnosis and course of action. Thus a physician can make definitive diagnosis based on clinical judgment and include the diagnosis as part of standard practice. In one embodiment, the step of entering the other data via the keyboard includes entering, by the physician, at least one CPT code. In one embodiment, the step of determining the diagnostic result includes specifying, by the physician, at least one ICD code.

In one embodiment the method includes use of at least one of a keyboard 118 and a display. In another embodiment, the method comprises the steps of providing a report generating device, for example, a printer 132 in communication with the computing device 112; and outputting a report with the computing device and printing the report with the printer. The report includes at least one of the BMI result, the BMI diagnosis, the weight data, at least a portion of the other data, the BMI, the medical condition and the course of action. Although a printer 132 has been described for printing the report the present invention is not limited in this regard as a report can be generated by the computing device 112 and provided on at least one of a display, a printer, an electronic storage means such as a floppy disc or a compact disc and an audio device.

In one embodiment, the method comprises the steps of transmitting at least one of the other data and the BMI to a physician; and the physician specifies at least one of a CPT code and an ICD code.

In one embodiment, the method further comprises the steps of providing a trending module 236 in communication with the computing device 212, a physician's office 201 and a health plan administrator 202. The trending module 236 comprises a data storage device 238. The method also includes the steps of storing successive inputs of at least one of the BMI result, the diagnostic result, the weight data, at least a portion of the other data, the BMI, the medical condition and the course of action in the data storage device; transmitting successive inputs of at least one of the BMI result, the diagnostic result, the weight data, at least a portion of the other data, the body mass index, the medical condition and the course of action in the data storage device to at least one of a physician and a health plan administrator; comparing the successive inputs of at least one of the weight data, at least a portion of the other data, the BMI, the recommended medical condition and the course of action, in the trending module 236; and updating at least one of the medical condition and the course of action including a recommendation from at least one of the physician and the health plan administrator.

In another embodiment the method comprise the steps of providing a certification module 340 in communication with the computing device 312; uploading security data into the certification module; entering certification data of an operator into the certification module via the keyboard 318; comparing the certification data to the security data; and enabling continued operation of the computing device if the certification data matches at least a portion of the security data. In one embodiment the method also includes the steps of enabling the reporting means to generate the report if the certification data matches at least an additional portion of the security data and generating the report.

In one embodiment, the method includes the step of installing the body mass diagnostic system in at least one of a laboratory and a physician's office and incorporated into infrastructure existing in healthcare systems which are used in physician offices and laboratory clinics and for which healthcare systems physicians have familiarity with and confidence in.

In another embodiment, the method includes the steps of providing an interface link 342 between the diagnostic module 328 and at least one external data source. Each of the at least one external data source comprising at least one of medical history, laboratory reports, health plan administrator's data, self insured employer groups, third party administrators, health insurance policies and incentive plans; downloading the supplementary information from the at least one external data source; and the diagnostic module analyzing the supplementary information.

In another embodiment, the method includes the step of providing the diagnostic module 328 comprising at least one of an obesity profile, an anorexia profile, a thyroid profile, a cardiac profile, lipid, panel, a blood chemistry profile, a cancer profile, a diabetes profile and a genetics profile.

In yet another embodiment the other data includes at least one of a height of the person, a waist measurement of the person, an age of the person, a gender of the person, an exercise program of the person, an athletic build of the person, body frame type, blood pressure any health conditions of the person, a date and a time.

In still another embodiment the method includes the steps of providing at least one tape measure 344; measuring a height of the person; and obtaining a waist measurement of the person.

Although the present invention has been disclosed and described with reference to certain embodiments thereof, it should be noted that other variations and modifications may be made, and it is intended that the following claims cover the variations and modifications within the true scope of the invention.

What is claimed is:

1. A body mass diagnostic system comprising:
input means for entry of at least one of weight data and other data;
a computing device in communication with said input means, said computing device comprising a body mass index module for generating a BMI result for evaluation by a physician and a diagnostic module for generating a diagnostic result based at least on said BMI result and direction from the physician for at least one patient to implement.

2. The body mass diagnostic system of claim 1 wherein said input means includes a scale for obtaining the weight data, a keyboard for entering at least one of the weight data and the other data for the at least one patient.

3. The body mass diagnostic system of claim 1 wherein said diagnostic module lists at least one CPT code for selection by the physician.

4. The body mass diagnostic system of claim 1 wherein said BMI result comprises at least one of a BMI, a normal operating BMI operating range, an indication of whether the BMI is within or outside of the normal operating BMI range, the weight data, health condition and at least a portion of the other data for the at least one patient.

5. The body mass diagnostic system of claim 1 wherein said diagnostic result includes at least one of a recommended diagnosis code, at least a portion of the other data, a recommended medical condition, a diagnosis of a medical condition, and a course of action for the at least one patient to implement.

6. The body mass diagnostic system of claim 1 wherein said recommended diagnosis code comprises an ICD code specified by the physician.

7. The body mass diagnostic system of claim 1 comprising:
reporting means in communication with said computing device; and
wherein said reporting means generates a report including at least one of the BMI result and the diagnostic result.

8. The body mass diagnostic system of claim 7 wherein said reporting means comprises at least one of a display, a printer, an electronic storage device and an audio device.

9. The body mass diagnostic system of claim 1 wherein said computing device further comprises:
a trending module comprising a data storage device for storing successive inputs of at least one of the BMI result and the diagnostic result taken over successive periods of time;
wherein said trending module transmits said successive inputs of at least one of the BMI result and the diagnostic result to at least one of the physician and a health plan administrator and said trending module compares a first of said successive inputs to a second of said successive inputs; and
wherein said trending module updates the diagnostic result, stores said updated diagnostic result in said storage device, and provides said BMI result and said updated diagnostic result to the at least one patient.

10. The body mass diagnostic system of claim 1 wherein an operator operates said input means to enter operator certification data and wherein said computing device further comprises:
a certification module comprising operator certification data and security data;
wherein said certification module compares said certification data to said security data; and
wherein when said certification data matches at least a portion of said security data, said certification module enables the operator to continue to use said computing device.

11. The body mass diagnostic system of claim 1, wherein said computing device further comprises:
an interface link between said computing device and at least one external data source for obtaining supplementary information therefrom;
wherein said supplementary information comprises at least one of medical history, laboratory reports, health plan administrator's data, self insured employer groups, third party administrators, health insurance policies and incentive plans; and
wherein said diagnostic module analyzes the supplementary information.

12. The body mass diagnostic system of claim 1, wherein said diagnostic module comprises at least one of an obesity profile, an anorexia profile, a thyroid profile, a cardiac profile, a body chemistry profile, a cancer profile, a diabetes profile and a genetics profile.

13. The body mass diagnostic system of claim 1, wherein said other data includes at least one of a height measurement of the person, a waist measurement of the person, an age of the person, gender of the person, an exercise program of the person, an athletic build of the person, body frame, a physician's orders, blood pressure, any health conditions of the person, a date and a time.

14. The body mass diagnostic system of claim 1 further comprising at least one length measuring means for determining at least one of a height measurement of the patient and a waist measurement of the person.

15. A method for diagnosing body mass comprising the steps of:
providing a body mass diagnostic system comprising input means and a computing device in communication with said input means, said computing device comprising a body mass index module and a diagnostic module;
obtaining weight data from a patient and transmitting the weight data to said computing device;
entering other data via said input means and transmitting the other data to said computing device;
calculating, by said body mass index module, a BMI result;
transmitting the BMI result to said diagnostic module and to a physician; and
determining a diagnostic result, based at least on said BMI result and input from the physician.

16. The method of step 15 wherein the step of entering other data via said input means includes entering, by the physician, at least one CPT code.

17. The method of step 15 wherein the step of determining a diagnostic result includes specifying, by the physician, at least one ICD code.

18. The method of step 15 comprising the steps of:
providing reporting means in communication with said computing device;
generating a report including at least one of the weight data, at least a portion of the other data, the body mass index, the medical condition and the course of action; and
outputting, by said reporting means, said report.

19. The method of claim 15 comprising the steps of:
providing a trending module within said computing device, said trending module in communication with at least one of a physician's office and a health plan administrator, said trending module comprising a data storage device; and
storing successive inputs of at least one of said BMI result and said diagnostic result in said data storage device;
transmitting successive inputs of at least one of said BMI result and said diagnostic result to at least one of the physician and the health plan administrator;
said trending module comparing said successive inputs of at least one of the weight data, at least a portion of the other data, the body mass index, the medical condition and the course of action; and
updating at least one of the medical condition and the course of action including a recommendation from at least one of the physician and the health plan administrator.

20. The method of claim 15 comprising the step of installing said body mass diagnostic system in at least one of a laboratory and a physician's office.

21. The method of claim 15 comprising the steps of:
providing a certification module in said computing device;
uploading security data into said certification module;
entering certification data of an operator into said certification module via said input means;
comparing said certification data to said security data; and
enabling continued operation of said computing device wherein when said certification data matches at least a portion of said security data.

22. The method of claim 15 including the steps of:
providing an interface link between said diagnostic module and at least one external data source, said at least one external data source comprising supplementary information including at least one of medical history, laboratory reports, health plan administrator's data, self insured employer groups, third party administrators, health insurance policies and incentive plans;
downloading the supplementary information from said at least one external data source; and
analyzing, by said diagnostic module, the supplementary information.

23. The method of claim 15 wherein said diagnostic module comprises at least one of an obesity profile, an anorexia profile, a thyroid profile, a cardiac profile, a body chemistry profile, a cancer profile, a diabetes profile and a genetics profile.
